(19) United States
(12) Patent Application Publication
(10) Pub. No.: US 2015/0290322 A1
(43) Pub. Date: Oct. 15, 2015

(54) SUSTAINED-RELEASE LIPID PRE-CONCENTRATE OF ANIONIC PHARMACOLOGICALLY ACTIVE SUBSTANCES AND PHARMACEUTICAL COMPOSITION COMPRISING THE SAME

(71) Applicant: CHONG KUN DANG PHARMACEUTICAL CORP., Seoul (KR)

(72) Inventors: Sang Phil Yoon, Gyeonggi-do (KR); Ki Seong Ko, Gyeonggi-do (KR); Eun Jeong Park, Gyeonggi-do (KR); Sung Joon Hong, Gyeonggi-do (KR); So Hyun Park, Gyeonggi-do (KR); Min Hyo Ki, Gyeonggi-do (KR)

(21) Appl. No.: 14/440,059

(22) PCT Filed: Dec. 27, 2013
(86) PCT No.: PCT/KR2013/012265

§ 371 Date: Apr. 30, 2015

(30) Foreign Application Priority Data
Dec. 28, 2012 (KR) 10-2012-0157582

(51) Int. Cl. A61K 47/24; A61K 31/439; A61K 31/69; A61K 9/00

(52) U.S. Cl. CPC A61K 47/24; A61K 9/0019; A61K 31/439; A61K 31/69

(57) ABSTRACT

Disclosed is a sustained-release lipid pre-concentrate, comprising: a) at least one liquid crystal former; b) at least one phospholipid; c) at least one liquid crystal hardener; and d) at least one bi- or multivalent metal salt, wherein the sustained-release pre-concentrate exists as a lipid liquid phase in the absence of aqueous fluid and forms into a liquid crystal upon exposure to aqueous fluid. The sustained-release lipid pre-concentrate is configured to enhance the sustained release of anionic pharmacologically active substances through ionic interaction between the bi- or multivalent metal salt and the anionic pharmacologically active substances.

### TECHNICAL FIELD

The present invention relates to a sustained release lipid pre-concentrate of anionic pharmacologically active substances, and a pharmaceutical composition comprising the same.

### BACKGROUND ART

Arising as promising dosage forms to reduce either side effects caused by multiple doses of pharmacologically active substances that are necessary to maintain the effective plasma concentration of the substance in blood stream for a specific period of time, or the administration frequency, sustained-release formulations have been extensively studied. A sustained-release formulation is of a drug delivery system (DDS) designed to release a single dose of a pharmacologically active substance at an effective concentration for a certain period of time.

PLGA [poly(lactic-co-glycolic acid)] is a representative of the currently used biodegradable materials which are approved for use in sustained release by the Food and Drug Administration (FDA). PLGA is a kind of copolymer in which lactic acid or lactide, and glycolic acid or glycolide are copolymerized at various ratios, and is described in U.S. Pat. No. 5,480,656 to allow for the sustained release of pharmacologically active substances by way of the degradation of PLGA into lactic acid and glycolic acid over a specific period of time in vivo. However, the acidic degradation products of PLGA induce inflammation, decreasing cell growth (K. Athanasiou, G. G. Niederauer and C. M. Agrawal, Biomaterials, 17, 93 (1996)). For sustained release, PLGA solid particles of 10˜100 micrometers in diameter, including a drug therein must be injected. The injection of the PLGA solid particles is accompanied by pain or inflammation. There is therefore a need for a novel sustained release formulation that supplies the effective plasma concentration of a pharmacologically active substance in blood stream for a prolonged period of time with improved patient compliance.

Previously, the present inventors introduced a sustained-release pre-concentrate comprising: a) at least one liquid crystal former; b) at least one phospholipid; and c) at least one liquid crystal hardener, which exists as a lipid liquid phase in the absence of aqueous fluid, and forms into a liquid crystal upon exposure to aqueous fluid.

When neutral or lipid-soluble pharmacologically active substances were applied thereto, the pre-concentrate introduced by the present inventors were found to release the pharmacologically active substances in a sustained release, with the maintenance of an effective plasma concentration for a long period of time. For anionic drugs or drugs with a net charge of (−), however, the pre-concentrate shows a high initial release rate, and a short maintenance time of effective plasma concentration, compared to neutral or lipid-soluble drugs.

There is therefore a method required for sustained release without an initial burst by which anionic drugs can be maintained at an effective concentration in vivo for a prolonged period of time.

Culminating in the present invention, intensive and thorough research of the present inventors into the sustained release formulation led to the findings that sustained-release a lipid pre-concentrate comprising a) at least one liquid crystal former, b) at least one phospholipid, c) at least one liquid crystal hardener, and d) at least one bi- or multivalent metal salt, exists as a lipid liquid phase in the absence of aqueous fluid and forms into a liquid crystal in aqueous fluid, with high in vivo safety and biodegradability, and that when associated with e) at least one anionic pharmacologically active substance, the pre-concentrate can release the active substance at an effective concentration for a long period of time.

Reference is now made to prior arts relevant to the present invention.

International Patent Publication No. WO 2005/117830 describes a pre-formulation comprising a low viscosity, non-liquid crystalline, mixture of: at least one neutral diacyl lipid and/or at least one tocopherol, at least one phospholipid, and at least one biocompatible, oxygen-containing, low viscosity organic solvent. International Patent Publication No. WO 2006/075124 discloses pre-formulations of a low viscosity mixture containing at least one diacyl glycerol, at least one phosphatidylcholine, at least one oxygen-containing organic solvent, and at least one somatostatin analogue. All these pre-formulations release the pharmacologically active substances in vivo for two weeks or longer, but, the organic solvents used are found to decrease the activity of some drugs (H. Ljusberg-Wahre, F. S. Nielse, 298, 328-332 (2005); H. Sah, Y. Bahl, Journal of Controlled Release 106, 51-61(2005)). Another different with the present invention is that bi- or multivalent metal salts are not essential components.

U.S. Pat. No. 7,731,947 discloses a composition comprising: a particle formulation comprising an interferon, sucrose, methionine, and a citrate buffer, and a suspending vehicle comprising a solvent such as benzyl benzoate, wherein the particle formulation is dispersed in the suspending vehicle. In one Example, it is described that phosphatidylcholine is dissolved together with vitamin E (tocopherol) in an organic solvent and is used to disperse the particle formulation therein. However, this composition is different from the transparent and filterable solution formulation of the present invention in that the composition is used to disperse solid particles and does not allow the formation of liquid crystals.

U.S. Pat. No. 7,871,642 discloses a method of preparing dispersions for delivering a pharmacologically active substance, comprising dispersing a homogeneous mixture of a phospholipid, a polyoxyethylene coemulsifier, triglyceride and ethanol in water, wherein the polyoxyethylene coemulsifier is selected from among polyethoxylated sorbitan fatty acid esters (polysorbate) and polyethoxylated vitamin E derivatives. However, Polyethoxylated sorbitan fatty acid esters and polyethoxylated vitamin E derivatives, derived by conjugating the hydrophilic polymer polyoxyethylene to sorbitan fatty acid ester and vitamin E, respectively, are quite different in structure from sorbitan fatty acid ester and vitamin E. They are usually used as hydrophilic surfactants utilizing the property of polyoxyethylene, which is different from the component of the present invention.

U.S. Pat. No. 5,888,533 discloses a flowable composition for forming a solid biodegradable implant in situ within a body, comprising: a non-polymeric, water-insoluble, biodegradable material; and a biocompatible, organic solvent that at least partially solubilizes the material and is miscible or dispersible in water or body fluids, and capable of diffusing-out or leaching from the composition into body fluid upon placement within a body, whereupon the non-polymeric material coagulates or precipitates to form the solid implant. In this composition, sterols, cholesteryl esters, fatty acids, fatty acid glycerides, sucrose fatty acid esters, sorbitan fatty acid esters, fatty alcohols, esters of fatty alcohols with fatty acids, anhydrides of fatty acids, phospholipids, lanolin, lanolin alcohols, and mixtures thereof are described as the non-polymeric material, and ethanol is used as the solvent. However, differences from the present invention reside in that this composition cannot form liquid crystals and is designed to form solid implants by simple coagulation or precipitation of water-insoluble materials and that a lot of the organic solvent is necessarily used.

International Patent Publication No. WO 2010/139278 discloses a preparation method of a drug-loaded oil-in-water emulsion containing phosphatidylcholine as a surfactant, and a-tocopherol acetate as an antioxidant. However, this composition does not form into a liquid crystal in aqueous fluid, and is further different from the present invention in terms of the use of phosphatidylcholine as a surfactant responsible for solubilizing into an oil phase or dispersing within a water phase, and α-tocopherol acetate as an antioxidant.

Korean Patent Publication No. 10-2011-0056042 discloses a tumor-targeting pharmaceutical composition in a nano-dispersion, comprising an anticancer drug as a pharmacologically active substance, a bi- or trivalent transition metal ion or alkaline earth metal ion, an oil, and hyaluronic acid or a salt thereof. It is further described that the oil may be α-tocopherol or a salt thereof while the surfactant is sorbitan monooleate. Because the composition has a final form of nano-particles which are obtained by precipitating the nano-dispersion, it is different from the composition of the present invention which forms into a liquid crystal. In addition, bi- or trivalent transition ions or alkaline earth metal ions serve to associate hyaluronic acid or a salt thereof onto the surface of the nanoparticles.

International Patent Publication No. WO 2005/048930 describes an injectable composition comprising a surfactant, a solvent, and a beneficial agent, wherein upon exposure to a hydrophilic environment, the surfactant and solvent form a viscous gel and the beneficial agent is dispersed or dissolved in the gel. As the surfactant which forms a viscous gel in a hydrophilic environment, phospholipids or PEGylated phospholipids are used while ethanol or tocopherol serve as the hydrophobic solvent. Thus, this composition which forms a viscous gel in a hydrophilic environment is different from the composition of the present invention which becomes a liquid crystal upon exposure to aqueous fluid.

International Patent Publication No. WO 2010/108934 discloses a vesicular drug delivery system comprising at least one lipid bilayer enclosing at least one aqueous cavity; at least one short interfering ribonucleic acid (siRNA) molecule contained within the aqueous cavity; and at least one hydrophobic drug substance embedded in the lipid bilayer, and optionally a pharmaceutically acceptable excipient selected from among cholesterol, polyethylene glycol (PEG) and tocopherol. However, phosphatidylcholine and the excipient tocopherol cannot form a liquid crystal upon exposure to aqueous fluid, which is different from the present invention.

In International Patent Publication No. WO 2005/049069, an injectable depot gel composition includes a gel vehicle comprising a bioerodible, biocompatible polymer and a water-immiscible solvent, and uses an excipient to modulate release profiles and stabilize a beneficial agent. Among the excipients are pH modifiers including inorganic salts, organic salts and combinations thereof, and an antioxidant including d-α-tocopherol acetate and dl-α-tocopherol acetate. However, bioerodible, biocompatible PLGA, which is the essential substance for the composition, is not found in the present invention. Another difference from the present invention resides in the use of a metal salt as a pH modifier, and tocopherol acetate as an antioxidant.

International Patent Publication No. WO 2005/110360 describes a lipid composition comprising at least one biologically active compound, a membrane lipid containing phosphatidylcholine, with a liquid crystal phase transition temperature below 40° C., at least one water miscible, pharmaceutically acceptable organic solvent, a pharmaceutically acceptable carrier liquid, and other additives suitable for injection purposes. When exposed to an aqueous environment, this composition is converted to a viscous lipid matrix in a liquid crystal state, thus enabling the gradual release of the biologically active compound. However, the substance that plays an important role in the composition is a membrane lipid, which is different from the liquid crystal former of the present invention.

International Patent Publication No. WO 2008/139804 introduces a low-molecular drug-containing nanoparticle having a negatively charged group which is produced by hydrophobizing a low-molecular drug having a negatively charged group with a metal ion, and re-acting the hydrophobized product with PLGA. However, a difference from the present invention is the use of an excess of organic solvent in the preparation of PLGA nanoparticles, and metal ions in the hydrophobization of drugs. In addition, this composition has limited applications only low-molecular negatively charged drugs and does not mention in vivo drug release behaviors at all.

### DISCLOSURE OF INVENTION

### Technical Problem

It is therefore an object of the present invention to provide a sustained release lipid pre-concentrate, based on phase transition from lipid liquid phase into liquid crystal, for allowing for the sustained release of anionic pharmacologically active substances, with an enhancement in sustained release by ionic interaction between bi- or multivalent metal salts and the anionic pharmacologically active substances.

It is another object of the present invention to provide a sustained release lipid pre-concentrate which maintained stability and biodegradability in spite of the presence of bi- or multivalent metal salts.

### Solution to Problem

In accordance with an aspect thereof, the present invention provides a sustained-release lipid pre-concentrate, comprising: a) at least one lipid crystal former; b) at least one phospholipid; c) at least one liquid crystal hardener; and d) at least one bi- or multivalent metal salt, which exists as a lipid liquid phase in the absence of aqueous fluid and forms into a liquid crystal upon exposure to aqueous fluid.

In accordance with another aspect thereof, the present invention provides a pharmaceutical composition comprising e) at least one anionic pharmacologically active substance plus the sustained-release lipid pre-concentrate in which the anionic pharmacologically active substance exhibits enhanced sustained release as a result of ionic interaction with the bi- or multivalent metal salt of the sustained-release lipid pre-concentrate.

Below, a detailed description will be given of each component.

a) Liquid Crystal Former

The liquid crystal former used in the present invention is responsible for the formation of non-lamellar liquid crystals, and may be selected from the group consisting of sorbitan unsaturated fatty acid ester, monoacyl glycerol, diacyl glycerol, and a combination thereof.

For use as a liquid crystal former in the present invention, the sorbitan unsaturated fatty acid ester preferably has two or more —OH (hydroxyl) groups in the polar head. This sorbitan unsaturated fatty acid ester may be represented by the following Chemical Formula 1. The compound of Chemical Formula 1 is sorbitan monoester where R¹═R²═OH, R³═R, and sorbitan diester where R¹═OH, R²═R³═R, R being an alkyl ester group of 4 to 30 carbon atoms with at least one unsaturated bond.

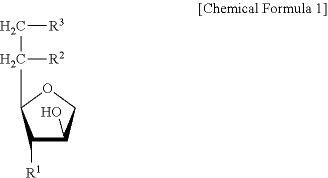

In detail, the sorbitan unsaturated fatty acid ester of the present invention may be obtained from whale oils and fish oils as well as vegetable oils and animal fats and oils. Preferable examples of vegetable oils include cacao butter, borage oil, unpolished rice oil, green tea oil, soybean oil, hempseed oil, sesame oil, cherry seed oil, rapeseed oil, poppy seed oil, pumpkin seed oil, grape seed oil, apricot kernel oil, coconut oil, camellia oil, evening primrose oil, sunflower seed oil, canola oil, pine nut oil, walnut oil, hazelnut oil, avocado oil, almond oil, peanut oil, jojoba oil, palm oil, castor oil, olive oil, corn oil, cottonseed oil, safflower seed oil, and primrose oil. Preferable examples of the animal fat and oil include milk fat, beef tallow, mammal oil, reptile oil, and bird oil. Preferably it may be selected from among sorbitan monoester, sorbitan sesquiester, sorbitan diester, which has fatty acid obtained from whale oils and fish oils, and a combination thereof.

Sorbitan monoester is a compound in which one fatty acid group is attached to sorbitan via an ester bond, and may be selected from among sorbitan monooleate, sorbitan monolinoleate, sorbitan monopalmitoleate, sorbitan monomyristoleate, and a combination thereof.

Sorbitan sesquiester is a compound in which 1.5 fatty acid groups, on average, are attached to sorbitan via an ester bond, and may be selected from among sorbitan sesquioleate, sorbitan sesquilinoleate, sorbitan sesquipalmitoleate, sorbitan sesquimyristoleate, and a combination thereof.

Sorbitan diester is a compound in which two fatty acid groups are attached to sorbitan via an ester bond, and may be selected from among sorbitan dioleate, sorbitan dilinoleate, sorbitan dipalmitoleate, sorbitan dimyristoleate, and a combination thereof.

For use in the present invention, sorbitan unsaturated fatty acid ester is preferably selected from sorbitan monooleate, sorbitan monolinoleate, sorbitan monopalmitoleate, sorbitan monomyristoleate, sorbitan sesquioleate, and a combination thereof.

Monoacyl glycrol, which can be used as a liquid crystal former in the present invention, consists of glycerine as the polar head and one fatty acid as a tail, with a linkage therebetween via an ester bond, while diacyl glycerol contains glycerine as the polar head with the same or different, two fatty acid tails attached thereto via ester bonds. Fatty acid groups, which attached to the mono- or diacyl glycerol via ester bonds used in the present invention, fatty acids may contain the same or different numbers of carbon atoms ranging from 4 to 30, and may independently be saturated or unsaturated. The fatty acid may be selected from among the group consisting of palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, and a combination thereof.

In detail, the monoacyl glycerol of the present invention may be selected from among glycerol monobutyrate, glycerol monobehenate, glycerol monocaprylate, glycerol monolaurate, glycerol monomethacrylate, glycerol monopalmitate, glycerol monostearate, glycerol monooleate, glycerol monolinoleate, glycerol monoarchidate, glycerol monoarchidonate, glycerol monoerucate, and a combination thereof. Preferable example of monoacyl glycerol is glycerol monooleate (GMO) represented by the following Chemical Formula 2.

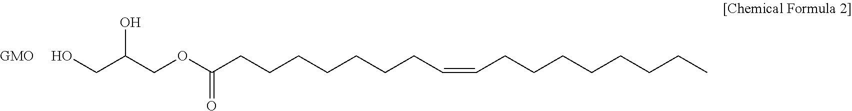

The diacyl glycerol of the present invention may be selected from among glycerol dibehenate, glycol dilaurate, glycerol dimethacrylate, glycerol dipalmitate, glycerol distearate, glycerol dioleate, glycerol dilinoleate, glycerol dierucate, glycerol dimyristate, glycerol diricinoleate, glycerol dipalmitoleate, and a combination thereof. Preferable example of diacyl glycerol is glycerol dioleate (GDO, represented by the following Chemical 3.

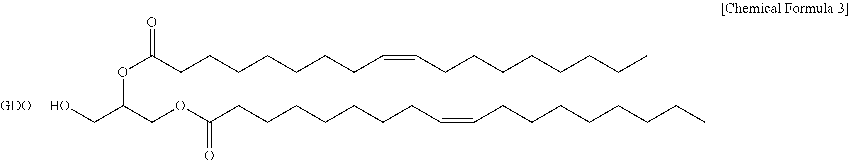

b) Phospholipid

Phospholipids are essential for the construction of lamellar structures, such as liposomes, in conventional techniques, but cannot form a non-lamellar phase structure, such as a liquid crystal, by themselves. However, phospholipids of the present invention participate in non-lamellar phase structures formed by the liquid crystal former and contribute to stabilizing of the liquid crystals.

The phospholipid of the present invention is derived from plants or animals, and contains a saturated or unsaturated alkyl ester group of 4 to 30 carbon atoms with a polar head. The phospholipid may be selected from among phosphatidylcholine, phosphatidylethanolamine, phosphatidylserine, phosphatidylglycerine, phosphatidylinositol, phosphatidic acid, sphingomyelin, and a combination thereof according to the structure of the polar head. In phospholipids, alkyl ester groups include saturated fatty acid esters such as mono- and dipalmitoyl, mono- and dimyristoyl, mono- and dilauryl, and mono- and distearyl, and unsaturated fatty acid chains such as mono- or dilinoleyl, mono- and dioleyl, mono- and dipalmitoleyl, and mono- and dimyristoleyl. Saturated and unsaturated fatty acid esters can coexist in phospholipids.

c) Liquid Crystal Hardener

The liquid crystal hardener of the present invention cannot form a non-lamellar structure, unlike the liquid crystal former, nor a lamellar structure such as liposome unlike phospholipids, by itself. However, the liquid crystal hardener participates in non-lamellar phase structures and contributes to enhance the ordered co-existence of oil and water by increasing the curvature of the non-lamellar structures. In the interests of this function, the liquid crystal hardener is advantageously required to have a highly limited polar moiety and a bulky non-polar moiety inside its molecular structure.

In practice, however, biocompatible molecules which are injectable into the body only via direct and repeated experiments can be selected as the liquid crystal hardener of the present invention. As a result, liquid crystal hardeners suitable for the composition of the present invention have molecular structures which are different from one another and thus cannot be elucidated as one molecular structure. The common structural feature observed by identification of all of the liquid crystal hardeners suitable for the composition of the present invention is that they are free of ionizable groups, such as carboxyl and amine groups, and have hydrophobic moieties comprising a bulky triacyl group with 15 to 40 carbon atoms or carbon ring structure.

The liquid crystal hardener of the present invention may be free of ionizable groups, such as carboxyl and amine groups, and have at most one hydroxyl and ester group as a weak polar head, with hydrophobic moieties including a bulky triacyl group with 20 to 40 carbon atoms or carbon ring structure. Preferable example of the liquid crystal hardener of the present invention may be selected from among, but not limited to, triglyceride, retinyl palmitate, tocopherol acetate, cholesterol, benzyl benzoate, ubiquinone, and a combination thereof. Preferably, the liquid crystal hardener may be selected from among tocopherol acetate, cholesterol, and a combination thereof.

d) Bi- or Multivalent Metal Salt

In the structure of liposomes or micelles containing phospholipids, metal ions with positive charges associate with negatively charged phosphate groups of phospholipids (Journal of Lipid Research 8 (1967) 227-233). In addition, the presence of metal salts alleviates repulsive power between negative charges of phosphate groups, increasing the tightness of the liposomal or micelle structure (Chemistry and Physics of Lipids 151 (2008) 1-9).

Partially or entirely forming ionic bonds with anionic pharmacologically active substances as well as the phosphate groups of phospholipids within the liquid crystal structure, the di- or multivalent metal salts of the present invention prevent the anionic pharmacologically active substances from rapidly escaping from the liquid crystal structure. Thanks to this ionic interaction, the metal ions can significantly reduce initial burst, and enhance the sustained-release of an anionic pharmacologically active substance. With reference to FIG. 1, ionic interaction between anionic pharmacologically active substances and bi- or multivalent metal salts within a liquid crystal structure is schematically represented.

In the di- or multivalent metal salts of the present invention, example of pharmaceutically acceptable metals include salts of aluminum, calcium, iron, magnesium, tin, titanium, and zinc, with preference for zinc, aluminum or calcium.

In detail, the di- or multivalent metal salt may be selected from among, but not limited to, aluminum carbonate, aluminum chloride, aluminum hydroxide, aluminum oxide, aluminum phosphate, aluminum sulfate, calcium bromide, calcium carbonate, calcium chloride, calcium hydroxide, calcium nitrate, calcium oxide, calcium phosphate, calcium silicate, calcium sulfate, calcium acetate, ferric chloride, ferric hydroxide, ferric oxide, ferric sulfate, magnesium carbonate, magnesium chloride, magnesium hydroxide, magnesium nitrate, magnesium oxide, magnesium phosphate, magnesium silicate, magnesium sulfate, stannous chloride, stannous fluoride, stannous hydroxide, stannous oxide, stannous sulfate, titanium dioxide, zinc carbonate, zinc chloride, zinc hydroxide, zinc nitrate, zinc oxide, zinc phosphate, zinc sulfate, zinc acetate, and a combination thereof.

Preferable example of the di- or multivalent metal salt may be selected from among aluminum chloride, aluminum hydroxide, aluminum phosphate, calcium bromide, calcium chloride, calcium hydroxide, calcium oxide, zinc carbonate, zinc chloride, zinc hydroxide, zinc acetate and a combination thereof.

e) Anionic Pharmacologically Active Substance

The term “anionic pharmacologically active substance,” as used herein, refers to a pharmacologically active substance negatively charged or with a net charge of (−).

The anionic pharmacologically active substance of the present invention may be in the form of at least one selected from among carboxylic acid, sulfinic acid, sulfonic acid, phosphonic acid, phosphoric acid, boronic acid, borinic acid, aromatic alcohol, imide or quaternary ammonium halide salts.

Concrete examples of the anionic pharmacologically active substance useful in the present invention include bortezomib, methotrexate, olopatadine, tiotropium, ipratropium, glycopyrronium, aclidinium, umeclidinium, trospium, alendronic acid, ibandronic acid, incadronic acid, pamidronic acid, risedronic acid, zoledronic acid, etidronic acid, clodronic acid, tiludronic acid, olpadronic acid, neridronic acid, diclofenac, levocabastine, indomethacin, ibuprofene, flurbiprofen, fenoprofen, ketoprofen, naproxene, diclofenac, etodolac, sulindac, tolmetin, salicylic acid, difiunisal, oxaprozin, tiagabine, gabapentin, ciprofloxacin, levofloxacin, fusidic acid, aminolevulinic acid, aminocaproic acid, isopropamide iodide, trihexethyl chloride, cephalexin, aspirin, indoprofen, levodopa, methyldopa, zomepirac, cefamandole, alclofenac, mefenamic acid, flufenamic acid, lisinopril, enalapril, enalaprilat, captopril, ramipril, fosinopril, benazepril, quinapril, temocapril, cilazapril, valsartan, valproic acid, cromoglicic acid, tranilast, pantothenic acid, metiazinic acid, fentiazac, fenbufen, pranoprofen, loxoprofen, dexibuprofen, alminoprofen, tiaprofenic acid, aceclofenac, nalidixic acid, azelaic acid, mycophenolic acid, leucovorin, ethacrynic acid, tranexamic acid, ursodeoxycholic acid, folic acid, meclofenamic acid, carbenicillin, rebamipide, cetirizine, fexofenadine, letosteine, probenecid, hopantenic acid, baclofen, furosemide, piretanide, methyldopa, pravastatin, liothyronine, levothyroxine, minodronic acid, P-aminosalicylic acid, gluconic acid, biotin, liraglutide, exenatide, taspoglutide, albiglutide, lixisenatide, interferon alpha, interferon beta, interferon gamma, glucagon-like peptides, adrenocorticotropic hormone, insulin and insulin-like growth factors, parathyroid hormone and its fragments, darbepoetin alpha, epoetin alpha, epoetin beta, epoetin delta, infliximab, insulin, glucagon, glucagon-like peptides, thyrotropin hormone, thyroid stimulating hormone, parathyroid hormone, calcitonin, adrenocorticotropic hormone (ACTH), follicle stimulating hormone, chorionic gonadotropin, gonadotropin releasing hormone, somatropin, GRF, lypressin, luteinizing hormone, interleukin, growth hormone, prostaglandin, platelet-derived growth factors (PDGF), keratinocyte growth factors (KGF), fibroblast growth factors (FGF), epidermal growth factors (EGF), transforming growth factor-α (TGF-α), transforming growth factor-β (TGF-β), erythropoietin (EPO), insulin-like growth factor-I (IGF-I), insuin-like growth factor-II (IGF-II), tumor necrosis factor-α (TNF-α), tumor necrosis factor-β (TNF-β), colony stimulating factor (CSF), vascular cell growth factor (VEGF), trombopoietin (TPO), stromal cell-derived factors (SDF), placenta growth factor (PIGF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone growth factor, bone morphogeneic proteins (BMF), coagulation factors, human pancreas hormone releasing factor, analogues and derivative thereof, pharmaceutically acceptable salts thereof, and a combination thereof.

Preferably, the anionic pharmacologically active substance may be selected from the group consisting of bortezomib, methotrexate, olopatadine, liraglutide, exenatide, taspoglutide, albiglutide, lixisenatide, interferon alpha, interferon beta, interferon gamma, tiotropium, ipratropium, glycopyrronium, aclidinium, umeclidinium, trospium, alendronic acid, ibandronic acid, incadronic acid, pamidronic acid, risedronic acid, zoledronic acid, etidronic acid, clodronic acid, tiludronic acid, olpadronic acid, neridronic acid, glucagon-like peptides, adrenocorticotropic hormone, insulin and insulin-like growth factors, parathyroid hormone and its fragments, darbepoetin alpha, epoetin alpha, epoetin beta, epoetin delta, diclofenac, levocabastine, indomethacin, ibuprofene, flurbiprofen, fenoprofen, ketoprofen, naproxene, diclofenac, etodolac, sulindac, tolmetin, salicylic acid, difiunisal, oxaprozin, tiagabine, gabapentin, ciprofloxacin, levofloxacin, fusidic acid, aminolevulinic acid, a pharmaceutically acceptable salt thereof, and a combination thereof.

More preferably, the anionic pharmacologically active substance may be selected from the group consisting of tiotropium, ipratropium, glycopyrronium, aclidinium, umeclidinium, trospium, pharmaceutically acceptable salts thereof, and a combination thereof.

It will be appreciated that the anionic pharmacologically active substance applicable to the sustained release lipid pre-concentrate of the present invention is not limited to the foregoing examples of drugs. So long as it is negatively charged, any pharmacologically active substance may be used in the present invention.

With regard to the pH of the composition of the present invention, no particular limitations are imparted if it falls within a typical physiologically acceptable range. As needed, a pH modifier may be used. It may be selected from among, but not limited to, hydrochloric acid, sulfuric acid, boric acid, phosphoric acid, acetic acid, sodium hydroxide, ethanolamine, diethanolamine, and triethanolamine.

As used herein, the term “aqueous fluid” is intended to include water and body fluids such as a mucosal solution, a tear, sweat, saliva, gastrointestinal fluid, extravascular fluid, extracellular fluid, interstitial fluid, and plasma. When exposed to aqueous fluid, the composition of the present invention undergoes transition from a lipid liquid phase to a liquid crystal phase with a semi-solid appearance. That is, the composition of the present invention is a pre-concentrate which exists as a lipid liquid state before application to the human body and shifts into a liquid crystal phase promising sustained release within the body.

The liquid crystals formed by the composition of the present invention have a non-lamellar phase structure in which oil and water are in an ordered mixture and arrangement without discrimination between inner and out phases. The ordered arrangement of oil and water renders the non-lamellar phase structure of a mesophase, which is a state of matter intermediate between liquid and solid. The pre-concentrate of the present invention is different from conventional compositions that form lamellar structures, such as micelles, emulsions, microemulsions, liposomes, and lipid bilayers, which have been widely used in designing pharmaceutical formulations. Such lamellar structures are in oil in water (o/w) or water in oil (w/o) type in which there is clear discrimination inner and out phases, and thus are different from the liquid crystals of the present invention.

Therefore, the term “liquid crystallization,” as used herein, refers to the formation of liquid crystals having a non-lamellar phase structure from the pre-concentrate upon exposure to aqueous fluid.

In the pre-concentrate of the present invention, the weight ratio between components of a) and b) is in a range of from 10:1 to 1:10, and preferably in a range of 5:1 to 1:5. The weight ratio of a)+b) to c) falls within the range of from 1,000:1 to 1:1, and preferably within the range of from 50:1 to 2:1. Turning to the weight ratio of a)+b)+c) to d), it ranges from 1,000:1 to 10:1, and preferably from 500:1 to 20:1. Given these weight ranges, the components efficiently guarantee the sustained release attributable to liquid crystals and the bi- or multivalent metal ion-induced improvement in sustained release.

Generally, the pharmaceutical composition of the present invention may comprise a weight ratio of a)+b)+c)+d) to e) in the range of from 10,000:1 to 1:1, which may vary depending on the kind of the pharmacologically active substance, the kind of formulation to be applied, desired release patterns, and the dose of the pharmacologically active substance required in the medical field.

The sustained release lipid pre-concentrate of the present invention may be prepared at room temperature from a) at least one liquid crystal former, b) at least one phospholipid, c) at least one liquid crystal hardener, and d) at least one bi- or multivalent metal salt, and if necessary, by heating or using a homogenizer. The homogenizer may be a high-pressure homogenizer, an ultrasonic homogenizer, a bead mill homogenizer, etc.

As described above, the sustained-release lipid pre-concentrate of the present invention may be a pharmaceutical composition which exists as a lipid liquid phase in the absence of aqueous fluid and forms into liquid crystals in the presence of aqueous fluid. As it turns to a pharmaceutical composition which can be applied to the body using a route selected from among injection, coating, dripping, padding, oral administration, and spraying, the pre-concentrate of the present invention may be preferably formulated into various dosage forms including injections, ointments, gels, lotions, capsules, tablets, solutions, suspensions, sprays, inhalants, eye drops, adhesives, and plaster and pressure sensitive adhesives, and more preferably into injections.

Particularly, when an injection route is taken, the pre-concentrate of the present invention may be administered by subcutaneous or intramuscular injection depending on the properties of the pharmacologically active substance used.

The pharmaceutical composition of the present invention may be preferably in the formulation form selected from among injections, ointments, gels, lotions, capsules, tablets, solutions, suspensions, sprays, inhalants, eye drops, adhesives, and plaster and pressure sensitive adhesives, and more preferably into injections.

The pharmaceutical composition of the present invention may be prepared by adding a pharmacologically active substance to the pre-concentrate of the present invention. As needed, heat or a homogenizer may be used in the preparation of the pharmaceutical composition of the present invention, but this is not a limiting factor to the present invention.

The dose of the pharmaceutical composition of the present invention adheres to the well-known dose of the pharmacologically active substance employed, and may vary depending on various factors including the patient's condition, age and sex. It may be administered orally or parenterally.

In accordance with a further aspect thereof, the present invention contemplates a method of maintaining pharmaceutical efficacy through the sustained release of a pharmacologically active substance by administering the pharmaceutical composition of the present invention to a mammal including a human, and the use of the pharmaceutical composition for the sustained release of a pharmacologically active substance.

### Advantageous Effects of Invention

As described hitherto, the sustained-release lipid pre-concentrate and the pharmaceutical composition according to the present invention, guarantee excellent sustained release of the pharmacologically active substance on the basis of ionic interaction between the bi- or multivalent metal salt and the anionic pharmacologically active substance within the liquid crystals formed.

### BRIEF DESCRIPTION OF DRAWINGS

FIG. 1 is a schematic view illustrating partial or entire ionic interaction between bi- or multivalent metal salts and anionic pharmacologically active substances within the sustained-release lipid pre-concentrate.

FIG. 2 shows in vivo biodegradability of the sustained-release lipid pre-concentrates of Examples 1 and 3, the pharmaceutical compositions of Examples 21 and 27, and the lipid pre-concentrates of Comparative Examples 3 and 5.

FIG. 3 shows in vivo drug release behaviors of the pharmacologically active substance (tiotropium bromide) of the compositions of Example 21 and Comparative Examples 21 and 29.

FIG. 4 shows in vivo drug release behaviors of the pharmacologically active substance (bortezomib) of the compositions of Example 26 and Comparative Example 22.

FIG. 5 shows phase change behaviors of the compositions of Example 4 and Comparative Example 22 and 27 upon exposure to aqueous fluid.

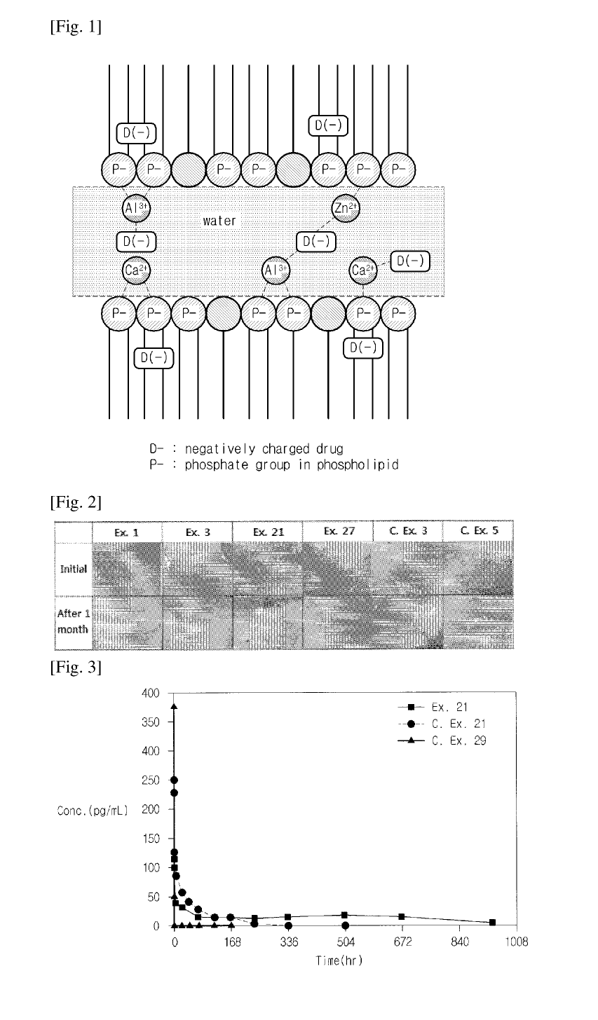

### MODE FOR THE INVENTION

A better understanding of the present invention may be obtained through the following examples which are set forth to illustrate, but are not to be construed as limiting the present invention.

The additives and excipients used in the present invention satisfied the requirements of the Korean Pharmacopoeia and were purchased from Aldrich, Lipoid, Croda, and Seppic.

### EXAMPLES 1 TO 20

### Preparation of Lipid Pre-Concentrates Containing Bi- or Multivalent Metal Salts

At the weight ratios given in Table 1, below, liquid crystal formers, phospholipids, liquid crystal hardeners, and bi- or multivalent metal salts were mixed, optionally in a solvent.

In Examples 1 to 20, the substances were homogenously mixed in a water bath maintained at 20˜75° C. using a homogenizer (PowerGen model 125, Fisher) for 0.5˜3 hrs at 1,000˜3,000 rpm. Then, the resulting lipid solutions were left at room temperature to come to thermal equilibrium at 25° C. before being loaded into 1 cc disposable syringes. The lipid solutions were injected into water (2 g of deionized water) to afford pre-concentrates containing metal salts of the present invention.

TABLE 1

| | Example | | | | | | | | | |
|---|---|---|---|---|---|---|---|---|---|---|
| (Unit: mg) | 1 | 2 | 3 | 4 | 5 | 6 | 7 | 8 | 9 | 10 |
| Sorbitan monooleate | 35 | 50 | 51 | 42 | 48 | | | | | |
| Sorbitan sesquioleate | | | | | | 35 | 50 | 51 | 42 | 48 |
| Glycerol monooleate | | | | | | | | | | |
| Glycerol dioleate | | | | | | | | | | |
| Phosphatidylcholine | 52 | 43 | | | 40.7 | 52 | 43 | | | 40.7 |
| Phosphatidylethanolamine | | | 34 | 45 | | | | 34 | 45 | |
| Triglyceride | | | | | | | | | | |
| Tocopherol acetate | 7 | | 7 | 7 | | 7 | | 7 | 7 | |
| Benzyl benzoate | | | | | 10 | | | | | 10 |
| Ubiquinone | | | | | 0.3 | | | | | 0.3 |
| Cholesterol | | 5 | | | | | 5 | | | |
| Aluminum chloride | 1 | 1 | 1 | | | 1 | 1 | 1 | | |
| Calcium chloride | | 1 | 1 | 1 | | | 1 | 1 | 1 | |
| Zinc acetate | | | 1 | | 1 | | | 1 | | 1 |
| Ethanol | 5 | | 5 | 5 | | 5 | | 5 | 5 | |
| Form in aqueous phase | Liquid crystal | | | | | | | | | |
| | Example | | | | | | | | | |
| (Unit: mg) | 11 | 12 | 13 | 14 | 15 | 16 | 17 | 18 | 19 | 20 |
| Sorbitan monooleate | | | | | | | | | | |
| Sorbitan sesquioleate | | | | | | | | | | |
| Glycerol monooleate | 35 | 50 | 51 | 42 | 48 | | | | | |
| Glycerol dioleate | | | | | | 35 | 50 | 51 | 42 | 48 |
| Phosphatidylcholine | 52 | 43 | | | 40.7 | 52 | 43 | | | 40.7 |
| Phosphatidylethanolamine | | | 34 | 45 | | | | 34 | 45 | |
| Triglyceride | | | | | | | | | | |
| Tocopherol acetate | 7 | | 7 | 7 | | 7 | | 7 | 7 | |
| Benzyl benzoate | | | | | 10 | | | | | 10 |
| Ubiquinone | | | | | 0.3 | | | | | 0.3 |
| Cholesterol | | 5 | | | | | 5 | | | |
| Aluminum chloride | 1 | 1 | 1 | | | 1 | 1 | 1 | | |
| Calcium chloride | | 1 | 1 | 1 | | | 1 | 1 | 1 | |
| Zinc acetate | | | 1 | | 1 | | | 1 | | 1 |
| Ethanol | 5 | | 5 | 5 | | 5 | | 5 | 5 | |

| | |
|---|---|
| Form in aqueous phase | Liquid crystal |

### EXAMPLES 21 TO 32

### Pharmaceutical Compositions with Pharmacologically Active Substances

Liquid crystal formers, phospholipids, liquid crystal hardeners, bi- or multivalent metal salts, and anionic pharmacologically active substances were mixed, at the weight ratios given in Table 2, below, optionally in solvents.

In Examples 21 to 32, the substances were homogeneously mixed in a water bath maintained at 20˜75° C. using a homogenizer (PowerGen model 125, Fisher) for 0.5˜3 hrs at 1,000˜3,000 rpm. The resulting lipid solutions were left at room temperature to come to thermal equilibrium at 25° C., followed by adding each of the pharmacologically active substances tiotropium bromide, ipratropium bromide, and bortezomib thereto. Then, the substances were homogenized for about 1˜5 hrs to afford pharmaceutical compositions in a solution phase.

TABLE 2

| | Example | | | | | | | | | | | |
|---|---|---|---|---|---|---|---|---|---|---|---|---|
| (Unit: mg) | 21 | 22 | 23 | 24 | 25 | 26 | 27 | 28 | 29 | 30 | 31 | 32 |
| Tiotropium | 0.1/ | 0.1/ | 0.1/ | | | | 0.1/ | 0.1/ | 0.1/ | 0.1/ | | |
| bromide | 0.2 | 0.2 | 0.2 | | | | 0.2 | 0.2 | 0.2 | 0.2 | | |
| Ipratropium | | | | 0.2/ | 0.2/ | | | | | | 0.2/ | |
| bromide | | | | 0.4 | 0.4 | | | | | | 0.4 | |
| Bortezomib | | | | | | 3 | | | | | | 3 |
| Sorbitan | 46 | 43 | | 45.6 | | 45 | 44 | | | | | |
| monooleate | | | | | | | | | | | | |
| Sorbitan | | | 55 | | 48.6 | | | 51 | | | | |
| sesquioleate | | | | | | | | | | | | |
| Glycerol | | | | | | | | | 44 | | 45.6 | |
| monooleate | | | | | | | | | | | | |
| Glycerol | | | | | | | | | | 43 | | 45 |
| dioleate | | | | | | | | | | | | |
| Phosphatidyl | 41 | 45 | | 45.7 | | 41 | 45 | | 45 | 44.8 | 46 | 41.8 |
| choline | | | | | | | | | | | | |
| Phosphatidyl | | | 43.7 | | 40 | | | 42 | | | | |
| ethanolamine | | | | | | | | | | | | |
| Tocopherol | 7 | | | | | 5 | | 5 | 5 | | | 3 |
| acetate | | | | | | | | | | | | |
| Benzyl | | 5 | | | 5 | | 5 | | | 5 | | |
| benzoate | | | | | | | | | | | | |
| Ubiquinone | | | 0.3 | 0.3 | | | | | | 0.2 | | 0.2 |
| Cholesterol | | | | 7 | | | | | | | 7 | |
| Aluminum | 1 | 1 | | 1 | | 1 | | 1 | 1 | 1 | | 1 |
| chloride | | | | | | | | | | | | |
| Calcium | | 1 | | | | | 1 | | | 1 | 1 | |
| chloride | | | | | | | | | | | | |
| Zinc acetate | | | 1 | | 1 | | | 1 | | | | 1 |
| Ethanol | 5 | 5 | | | 5 | 5 | 5 | | 5 | 5 | | 5 |

### COMPARATIVE EXAMPLES 1 TO 20

### Preparation of Pre-Concentrates Devoid of Bi- or Multivalent Metal Salts

At the weight ratios given in Table 3, below, liquid crystal formers, phospholipids, and liquid crystal hardeners were mixed in a solvent.

In Comparative Examples 1 to 20, the substances were mixed in a water bath maintained at 20˜75° C. using a homogenizer (PowerGen model 125, Fisher) for about 0.5˜3 hrs at 1,000˜3,000 rpm. Then, the resulting lipid solutions were left at room temperature to come to thermal equilibrium at 25° C. before being loaded into 1 cc disposable syringes. The lipid solutions were injected into water (2 g of deionized water) to afford pre-concentrates according to Comparative Examples 1 to 20.

TABLE 3

| | Comparative Example | | | | | | | | | |
|---|---|---|---|---|---|---|---|---|---|---|
| (Unit: mg) | 1 | 2 | 3 | 4 | 5 | 6 | 7 | 8 | 9 | 10 |
| Sorbitan monooleate | 40 | 50 | 40 | 55 | 40 | | | | | |
| Sorbitan sesquioleate | | | | | | 35 | 50 | 45 | 45 | 40 |
| Glycerol monooleate | | | | | | | | | | |
| Glycerol dioleate | | | | | | | | | | |
| Phosphatidylcholine | 55 | | 48 | 40 | 48 | 39.7 | | 35 | 48 | 48 |
| Phosphatidylethanolamine | | 40 | | | | | 40 | | | |
| Triglyceride | 4.7 | | | | | 25 | | | | |
| Tocopherol acetate | | 10 | | 5 | 7 | | 10 | | 7 | 7 |
| Benzyl benzoate | | | 7 | | | | | | | |
| Ubiquinone | 0.3 | | | | | 0.3 | | | | |
| Cholesterol | | | | | | | | 15 | | |
| Ethanol | | | 5 | | 5 | | | 5 | | 5 |

| Form in aqueous phase | Liquid crystal | | | | | | | | | |
|---|---|---|---|---|---|---|---|---|---|---|
| | Comparative Example | | | | | | | | | |
| (Unit: mg) | 11 | 12 | 13 | 14 | 15 | 16 | 17 | 18 | 19 | 20 |
| Sorbitan monooleate | | | | | | | | | | |
| Sorbitan sesquioleate | | | | | | | | | | |
| Glycerol monooleate | 40 | 50 | 40 | 55 | 40 | | | | | |
| Glycerol dioleate | | | | | | 35 | 50 | 45 | 45 | 40 |
| Phosphatidylcholine | 54.7 | | 48 | 40 | 47.7 | 40 | | 35 | 48 | 50 |
| Phosphatidylethanolamine | | 40 | | | | | 40 | | | |
| Triglyceride | 5 | | | | | 25 | | | | |
| Tocopherol acetate | | 10 | | 5 | 7 | | 10 | | 7 | 5 |
| Benzyl benzoate | | | 7 | | | | | | | |
| Ubiquinone | 0.3 | | | | | 0.3 | | | | |
| Cholesterol | | | | | | | | 15 | | |
| Ethanol | | | 5 | 5 | | | | 5 | | 5 |

| | |
|---|---|
| Form in aqueous phase | Liquid crystal |

### COMPARATIVE EXAMPLES 21 TO 26

### Preparation of Pharmaceutical Compositions Devoid of Bi- or Multivalent Metal Salts

Liquid crystal formers, phospholipids, liquid crystal hardeners and anionic pharmacologically active substances were mixed at the weight ratios given in Table 4, below, optionally in a solvent.

In Comparative Examples 21 to 26, the substances were homogeneously mixed in a water bath maintained at 20˜75° C. using a homogenizer (PowerGen model 125, Fisher) for about 0.5˜3 hrs at 1,000˜3,000 rpm. The resulting lipid solutions were left at room temperature to come to thermal equilibrium at 25° C., followed by adding each of the pharmacologically active substances tiotropium bromide, ipratropium bromide, and bortezomib thereto. Then, the substances were homogenized for about 1˜5 hrs to afford pharmaceutical compositions in a solution phase.

TABLE 4

| | Comparative Example | | | | | |
|---|---|---|---|---|---|---|
| (Unit: mg) | 21 | 22 | 23 | 24 | 25 | 26 |
| Tiotropium bromide | 0.1/ | | | 0.1/ | | |
| | 0.2 | | | 0.2 | | |
| Ipratropium bromide | | | 0.2/ | | | 0.2/ |
| | | | 0.4 | | | 0.4 |
| Bortezomib | | 3 | | | 3 | |
| Sorbitan monooleate | 46 | 45 | | | | |
| Sorbitan sesquioleate | | | 45.6 | | | |
| Glycerol monooleate | | | | 46 | 51 | 45.6 |
| Glycerol dioleate | | | | | | |
| Phosphatidylcholine | 42 | 42 | | 46.8 | | 46 |
| Phosphatidylethanolamine | | | 46 | | 36 | |
| Tocopherol acetate | 5 | 5 | 5 | | 5 | 5 |
| Benzyl benzoate | | | 2 | | | |
| Ubiquinone | | | | 0.2 | | |
| Cholesterol | 2 | | | 2 | | |
| Ethanol | 5 | 5 | 1 | 5 | 5 | 1 |

### COMPARATIVE EXAMPLES 27 AND 28

### Preparation of Pre-Concentrates without Liquid Crystal Former

Pre-concentrates of Comparative Examples 27 and 28 were prepared by homogenously mixing polyoxyethylene sorbitan monooleate, phosphatidylcholine, and tocopherol acetate in a water bath maintained at 20˜75° C. using a homogenizer (PowerGen model 125, Fisher) for about 0.5˜3 hrs at 1,000˜3,000 rpm. Here, polyoxyethylene sorbitan monooleate has a polyoxyethylene group substituted for an —OH group on the sorbitan polar head and is different from sorbitan monooleate, used in the present invention. Polyoxyethylene sorbitan monooleate is generally used as a hydrophilic surfactant.

TABLE 5

| | C. Example | |
|---|---|---|
| (Unit mg) | 27 | 28 |
| Polyoxyethylene | 60 | 60 |
| sorbitan monooleate | | |
| Tocopherol | — | — |
| Tocopherol acetate | 10 | 5 |
| Phosphatidyl choline | 30 | 30 |
| Ethanol | — | 5 |

### COMPARATIVE EXAMPLES 29 AND 30

### Formulations of Anionic Pharmacologically Active Substances Unloaded to the Pre-Concentrated

For the formulation of Comparative Example 29, 2.2 μg of tiotropium bromide was added to 1 mL of physiological saline, followed by homogenization at room temperature.

The formulation of Comparative Example 30 was prepared by dissolving 5 mg of bortezomib in a mixture of 7 mL of physiological saline and 300 μl of ethanol at room temperature.

### EXPERIMENTAL EXAMPLE 11

### Assay for In Vitro Safety

A cytotoxic test was carried out using an Extraction Colony Assay to examine the compositions of the present invention for in vitro safety.

In 18 mL of Eagle's Minimal Essential Media (EMEM) supplemented with 10% fetal bovine serum was extracted 2 g of each of the compositions of Examples 1, 5, 21, and 27, and Comparative Examples 3 and 5. L929 cells (mouse fibroblast, American Type Culture Collection) were seeded at a density of 1×10² cells/well into 6-well plates, and stabilized for 24 hrs at 37° C. in a 5% CO₂ humidified incubator. The extracts were diluted in EMEM (0, 5, 25, 50%) and then placed in an amount of 2 mL/well in contact with the stabilized L929 cells.

After incubation for 7 days at 37° C. in a 5% CO₂ humidified incubator, the cells were fixed with a 10% formalin solution and stained with a Giemsa solution to count colonies. The results are summarized in Table 6, below.

TABLE 6

| | Relative colony formation rates(%)* | | | | | |
|---|---|---|---|---|---|---|
| Extract Medium | | | | | C. | C. |
| (v/v) % ** | Ex. 1 | Ex. 5 | Ex. 21 | Ex. 27 | Ex. 3 | Ex. 5 |
| 0% Medium (control) | 100.0 | 100.0 | 100.0 | 100.0 | 100.0 | 100.0 |
| 5% Medium | 97.7 | 95.5 | 95.2 | 93.4 | 90.6 | 91.2 |
| 25% Medium | 63.4 | 71.8 | 67.1 | 72.8 | 73.5 | 77.3 |
| 50% Medium | 11.1 | 18.3 | 12.2 | 13.7 | 12.5 | 14.5 |

*Relative colony formation rates (%) = Number of Colonies on Test Medium/Number of Colonies on 0% Medium × 100 (%)
** Extract Medium % = Extract Medium/(Diluted Medium + Extract Medium) × 100 (%)

As is understood from data of Table 6, the groups of Comparative Examples 3 and 5 grew at normal rates in each of the diluted media (5%, 25%, and 50%), with observation of similarity in growth rate between groups of Examples 1, 5, 21 and 27, and Comparative Examples 3 and 5. Accordingly, the lipid pre-concentrate and the pharmaceutical composition of the present invention were demonstrated to be highly safe to the body.

### EXPERIMENTAL EXAMPLE 2

### Assay for In Vivo Biodegradability

The compositions of the present invention were evaluated for in vivo biodegradability as follows.

Each of the compositions of Examples 1, 3, 21 and 27 was subcutaneously injected at a dose of 300 mg into the back of SD rats, and monitored for a predetermined period of time. For comparison, the compositions of Comparative Examples 3 and 5 were tested in the same manner. The injection sites were photographed one month after injection, and are shown in FIG. 2.

One month after injection, as can be seen in FIG. 2, the liquid crystal gel volumes were reduced to about ⅓ to ⅔ of the initial volumes in the groups of Comparative Examples 3 and 5, indicating the biodegradation of the compositions.

Likewise, the SD rats administered with the compositions of Examples 1, 3, 21 and 27 had the swelled tissues volumes reduced to ⅓ to ⅔ of the initial volumes one month after injection. Accordingly, the compositions of the present invention can degrade in vivo, to a degree similar to those of Comparative Examples 3 and 5.

For reference, PLGA [poly(lactic-co-glycolic acid)], a conventional widely used matrix for sustained release, is known to remain undegraded for as long as 2-3 months.

Hence, the lipid pre-concentrate comprising a bi- or multivalent metal salt of the present invention exhibited biodegradability similar to that of the compositions devoid of the metal salts, and overcomes the drawback of conventional sustained-release formulations that the carriers remain in the body for a long period of time even after the completion of drug release.

### EXPERIMENTAL EXAMPLE 3

### In Vivo Test for Sustained Release of Tiotropium Bromide

Drug release behaviors of tiotropium bromide from the compositions of the present invention were examined in vivo in the following test.

Using a disposable syringe, the composition of Example 21 was subcutaneously injected at a tiopropium bromide dose of 0.4 mg/kg into the back of 6 SD rats (male), 9 weeks old, with an average body weight of 300 g.

Tiotropium concentrations in plasma samples taken from the SD rats were analyzed using LC-MS/MS (liquid chromatography-tandem mass spectrometry) to draw PK profiles (pharmacokinetic profiles). The PK profiles in the SD rats are shown in FIG. 3.

For comparison of PK profiles, the composition of Comparative Example 29 was injected at a tiptropium bromide dose of 0.01 mg/kg subcutaneously to the back while the composition of Comparative Example 21, which was devoid of a bi- or multivalent metal salt, was applied at a tiotropium bromide dose of 0.4 mg/kg to the back by subcutaneous injection. The amount of the composition of Comparative Example 29 was one dose per day that is 30-fold lower than the dose of the sustained release formulation.

As can be seen in FIG. 3, the composition of Example 21 was significantly lower in initial burst, and exhibited higher sustained release, compared to the composition of Comparative Example 21, which lacked bi- or multivalent metal salts.

### EXPERIMENTAL EXAMPLE 4

### In Vivo Test for Sustained Release of Bortezomib

Drug release behaviors of bortezomib from the compositions of the present invention were examined in vivo in the following test. Using a disposable syringe, the composition of Example 26 was subcutaneously injected at a bortezomib dose of 0.6 mg/kg into the back of 6 SD rats (male), 9 weeks old, with an average body weight of 300 g.

Bortezomib concentrations in plasma samples taken from the SD rats were analyzed using LC-MS/MS (liquid chromatography-tandem mass spectrometry) to draw PK profiles (pharmacokinetic profiles). The PK profiles in the SD rats are shown in FIG. 4. In order to examine the effect of bi- or multivalent metal salts on sustained release, the composition of Comparative Example 22, which lacked bi- or multivalent metal salts, was injected at a bortezomib dose of 0.6 mg/kg subcutaneously to the back.

As can be seen in FIG. 4, the composition of Example 26 was significantly lower in initial burst, compared to the composition of Comparative Example 22, which lacked bi- or multivalent metal salts, and maintained effective concentrations, showing high sustained release.

### EXPERIMENTAL EXAMPLE 5

### Formation of Liquid Crystal in Aqueous Fluid

The composition of the present invention was evaluated for ability to form liquid crystal in an aqueous fluid as follows.

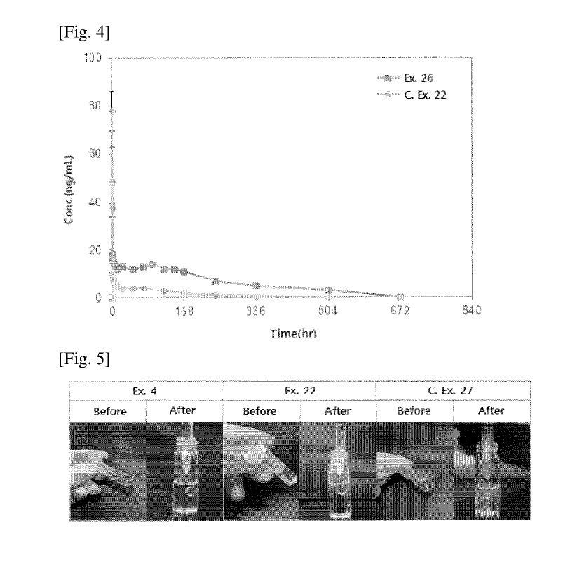

After being loaded into syringes, compositions of Examples 4 and 22 and Comparative Example 27 were dripped into 2 g of PBS (pH 7.4, and the results are shown in FIG. 5.

Both the compositions of Examples 4 and 22 were observed to exist as a lipid liquid phase in the absence of aqueous fluid before injection, but formed into liquid crystal after exposure to aqueous fluid. The composition of Comparative Example 27, based on polyoxyethylene sorbitan unsaturated fatty acid ester (polyoxyethylene sorbitan monooleate) was in the form of a liquid phase in the absence of aqueous fluid, and did not form into a liquid crystal after injection to aqueous fluid, but was dispersed in aqueous fluid. Accordingly, the sustained release composition of the present invention can rapidly shift from a liquid phase in the absence of aqueous fluid to a liquid crystal phase upon exposure to aqueous fluid, an in vivo environment, so that it can be applied to the sustained release formulation of medicinal agents.

Within the liquid crystals, there are a great number of bicontinuous water channels of nano size (below 20 nm) that resemble the Moebius strip. The water channels are surrounded with bicontinuous lipid layers. Thus, once a lipid composition forms into a liquid crystal in a semi-solid phase, a pharmacologically active substance can be released from the liquid crystal structure only after it has passed through numerous water channels and lipid layers, which enhances the sustained release effect of a pharmacologically active substance.

1. A sustained-release lipid pre-concentrate, comprising:
a) at least one liquid crystal former;
b) at least one phospholipid;
c) at least one liquid crystal hardener; and
d) at least one bi- or multivalent metal salt,
wherein the sustained-release pre-concentrate exists as a lipid liquid phase in the absence of aqueous fluid and forms into a liquid crystal upon exposure to aqueous fluid.

2. The sustained-release lipid pre-concentrate of claim 1, wherein the liquid phase former is selected from the group consisting of sorbitan unsaturated fatty acid ester, monoacyl glycerol, diacyl glycerol, and a combination thereof.

3. The sustained-release lipid pre-concentrate of claim 2, wherein the sorbitan unsaturated fatty acid ester has two or more —OH (hydroxyl) groups in the polar head.

4. The sustained-release lipid pre-concentrate of claim 2, wherein the sorbitan unsaturated fatty acid ester is selected from the group consisting of sorbitan monooleate, sorbitan monolinoleate, sorbitan monopalmitoleate, sorbitan monomyristoleate, sorbitan sesquioleate, sorbitan sesquilinoleate, sorbitan sesquipalmitoleate, sorbitan sesquimyristoleate, sorbitan dioleate, sorbitan dilinoleate, sorbitan dipalmitoleate, sorbitan dimyristoleate, and a combination thereof.

5. The sustained-release lipid pre-concentrate of claim 2, wherein the sorbitan unsaturated fatty acid ester is selected from the group consisting of sorbitan monooleate, sorbitan monolinoleate, sorbitan monopalmitoleate, sorbitan monomyristoleate, sorbitan sesquioleate, and a combination thereof.

6. The sustained-release lipid pre-concentrate of claim 2, wherein the monoacyl glycerol has a polar head consisting of glycerine, with a fatty acid tail attached thereto via an ester bond.

7. The sustained-release lipid pre-concentrate of claim 2, wherein the diacyl glycerol has a polar head consisting of glycerine, with two fatty acid tails attached thereto via respective ester bonds, said two fatty acid tails being the same or different from each other.

8. The sustained-release lipid pre-concentrate of claim 2, wherein the fatty acid groups attached to the monoacyl glycerol or the diacyl glycerol via ester bonds contains 4 to 30 carbon atoms, and is selected from the group consisting of palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, and a combination thereof.

9. The sustained-release lipid pre-concentrate of claim 2, wherein the monoacyl glycerol is selected from the group consisting of glycerol monobutyrate, glycerol monobehenate, glycerol monocaprylate, glycerol monolaurate, glycerol monomethacrylate, glycerol monopalmitate, glycerol monostearate, glycerol monooleate, glycerol monolinoleate, glycerol monoarchidate, glycerol monoarchidonate, glycerol monoerucate, and a combination thereof.

10. The sustained-release lipid pre-concentrate of claim 2, wherein the monoacyl glycerol is glycerol monooleate (GMO).

11. The sustained-release lipid pre-concentrate of claim 2, wherein the diacyl glycerol is selected from the group consisting of glycerol dibehenate, glycol dilaurate, glycerol dimethacrylate, glycerol dipalmitate, glycerol distearate, glycerol dioleate, glycerol dilinoleate, glycerol dierucate, glycerol dimyristate, glycerol diricinoleate, glycerol dipalmitoleate, and a combination thereof.

12. The sustained-release lipid pre-concentrate of claim 2, wherein the diacyl glycerol is glycerol dioleate (GDO).

13. The sustained-release lipid pre-concentrate of claim 1, wherein the phospholipid is selected from the group consisting of phosphatidylcholine, phosphatidylethanolamine, phosphatidylserine, phosphatidylglycerine, phosphatidylinositol, phosphatidic acid, sphingomyelin, and a combination thereof, having saturated or unsaturated carbon atoms in the range of 4 to 30.

14. The sustained-release lipid pre-concentrate of claim 1, wherein the liquid crystal hardener is free of an ionizable group and its hydrophobic moiety has a triacyl group with 15 to 40 carbon atoms or a carbon ring structure.

15. The sustained-release lipid pre-concentrate of claim 1, wherein the liquid crystal hardener is selected from the group consisting of triglyceride, retinyl palmitate, tocopherol acetate, cholesterol, benzyl benzoate, ubiquinone, and a combination thereof.

16. The sustained-release lipid pre-concentrate of claim 1, wherein the liquid crystal hardener is selected from the group consisting of tocopherol acetate, cholesterol, and a combination thereof.

17. The sustained-release lipid pre-concentrate of claim 1, wherein the metal of the bi- or multivalent metal salt is selected from the group consisting of aluminum, calcium, iron, magnesium, tin, titanium and zinc.

18. The sustained-release lipid pre-concentrate of claim 1, wherein the metal of the bi- or multivalent metal salt is selected from the group consisting of aluminum, calcium, and zinc.

19. The sustained-release lipid pre-concentrate of claim 1, wherein a weight ratio of a) to b) ranges from 10:1 to 1:10.

20. The sustained-release lipid pre-concentrate of claim 1, wherein a weight ratio of a)+b) to c) ranges from 1,000:1 to 1:1.

21. The sustained-release lipid pre-concentrate of claim 1, wherein a weight ratio of a)+b)+c) to d) ranges from 10,000:1 to 10:1.

22. A pharmaceutical composition, comprising:
the sustained-release lipid pre-concentrate of claim 1; and
e) at least one anionic pharmacologically active substance,
wherein the bi- or multivalent metal salt of the sustained-release pre-concentrate enhances the sustained release of the anionic pharmacologically active substance by forming an ionic bond with the anionic pharmacologically active substance.

23. The pharmaceutical composition of claim 22, wherein the anionic pharmacologically active substance is selected from the group consisting of pharmacologically active substance having at least one structure of a carboxylic acid, a sulfinic acid, a sulfonic acid, a phosphonic acid, a phosphoric acid, a boronic acid, a borinic acid, an aromatic alcohol, an imide or quaternary ammonium halide salts, a pharmaceutically acceptable salt thereof, and a combination thereof.

24. The pharmaceutical composition of claim 22, wherein the anionic pharmacologically active substance is selected from the group consisting of bortezomib, methotrexate, olopatadine, liraglutide, exenatide, taspoglutide, albiglutide, lixisenatide, interferon alpha, interferon beta, interferon gamma, tiotropium, ipratropium, glycopyrronium, aclidinium, umeclidinium, trospium, alendronic acid, ibandronic acid, incadronic acid, pamidronic acid, risedronic acid, zoledronic acid, etidronic acid, clodronic acid, tiludronic acid, olpadronic acid, neridronic acid, glucagon-like peptides, adrenocorticotropic hormone, insulin and insulin-like growth factors, parathyroid hormone and its fragments, darbepoetin alpha, epoetin alpha, epoetin beta, epoetin delta, diclofenac, levocabastine, indomethacin, ibuprofene, flurbiprofen, fenoprofen, ketoprofen, naproxene, diclofenac, etodolac, sulindac, tolmetin, salicylic acid, difiunisal, oxaprozin, tiagabine, gabapentin, ciprofloxacin, levofloxacin, fusidic acid, aminolevulinic acid, a pharmaceutically acceptable salt thereof, and a combination thereof.

25. The pharmaceutical composition of claim 22, wherein the anionic pharmacologically active substance is selected from the group consisting of tiotropium, ipratropium, glycopyrronium, aclidinium, umeclidinium, trospium, a pharmaceutically acceptable salt thereof, and a combination thereof.

26. The pharmaceutical composition of claim 22, wherein a weight ratio of a)+b)+c)+d) to e) ranges from 10,000:1 to 2:1.

27. The pharmaceutical composition of claim 2, that is formulated into a dosage form selected from among an injection, a ointment, a gel, a lotion, a capsule, a tablet, a solution, a suspension, a spray, an inhalant, an eye drop, an adhesive, and a plaster and pressure sensitive adhesive.

28. The pharmaceutical composition of claim 27, wherein the dosage form is an injection.
